(19) United States
(12) Patent Application Publication
(10) Pub. No.: US 2021/0052260 A1
(43) Pub. Date: Feb. 25, 2021

(54) HAND STABILIZING DEVICES

(71) Applicant: UNIVERSITY OF FLORIDA RESEARCH FOUNDATION, INCORPORATED, Gainesville, FL (US)

(72) Inventors: Abigail ESTELLE, Jacksonville, FL (US); Laurent GANRY, Jacksonville, FL (US)

(73) Assignee: University of Florida Research Foundation, Incorporated, Gainesville, FL (US)

(21) Appl. No.: 16/979,274

(22) PCT Filed: Mar. 11, 2019
(86) PCT No.: PCT/US2019/021702

§ 371 Date: Sep. 9, 2020

Related U.S. Application Data
(60) Provisional application No. 62/641,002, filed on Mar. 9, 2018.

(51) Int. Cl. A61B 17/00

(52) U.S. Cl. CPC A61B 17/00; A61B 2017/00862; A61B 2017/00438; A61B 2017/00424

(57) ABSTRACT

Disclosed herein is a unique surgical device that assists medical practitioners to stabilize and support their hand while conducting a surgical procedure. Specifically exemplified is a hand stabilizing device that includes a stem, having a shaft, a bent portion, and a base portion. The device may also be flexible. The device serves to support and stabilize the hand during a surgical procedure.

### FIELD

The disclosure relates generally to hand stabilizers, and more specifically to hand stabilizers for surgical procedures.

### BACKGROUND

Humans have limited ability and endurance to digitally manipulate precision tools such as surgical instruments or even artistic tools. A need exists in a variety of endeavors for convenient, unobtrusive hand stabilizers that improve accuracy, minimize hand tremors, and combat fatigue.

When performed by hand microsurgery, supermicrosurgery, microanastomosis, or reanastomosis and various other medical or surgical procedures or treatments may require fine-motor skills and an accuracy of micro-movements that may be difficult for any surgeon to execute. The movements required may be particularly difficult for surgeons-in-training or for surgeons with a tremor. Fatigue and discomfort may result when executing the required movements, for example in the muscles used to stabilize the surgeon's hands and forearms.

For at least these reasons, a need exists for a tool, a device, or a method to assist surgeons in performing such delicate procedures and treatments with consistency and accuracy without limiting free movement; flexibility; extended reach from any angle; the ability to quickly and easily re-position the wrist and forearm; and to grip, to operate, and to handle all surgical tools without interference.

### BRIEF DESCRIPTION OF THE DRAWINGS

Features, aspects, and advantages of the present disclosure will become better understood with reference to the following description and appended claims, and accompanying drawings where:

FIG. 1: shows a schematic diagram of a device according to various embodiments;

FIG. 2: shows a schematic diagram of a device according to various embodiments;

FIG. 3: shows a schematic diagram of a device according to various embodiments, being utilized by a surgeon as a surgical stabilizer in a first position;

FIG. 4: shows a schematic diagram of the device, as shown in FIG. 3, being utilized by a surgeon in a second position;

FIG. 5: shows a schematic perspective diagram of a device according to various embodiments; and

FIG. 6: shows a schematic perspective diagram of a device according to various embodiments.

The various embodiments disclosed herein are not limited to the arrangements and instrumentalities exemplified by the drawings.

### DETAILED DESCRIPTION

Disclosed herein is a unique surgical device that assists medical practitioners to stabilize and support their hand while conducting a surgical procedure. In a specific embodiment, disclosed is a hand stabilizing device that includes a stem, having a shaft, a bent portion, and a base portion. The shaft extends from a first end of the stem to the bent portion, and the base portion extending from a second end of the stem to the bent portion. The device further includes a first grip, associated at the first end of the stem, wherein the first grip defines an annular aperture. In an alternative embodiment, the hand stabilizing device may further include a second grip affixed to the first grip, wherein the second grip defines a second annular aperture.

The base portion of the device may extend laterally relative to the shaft, such that the base portion is laterally wider than the shaft. In a specific embodiment the base portion is laterally wider than the shaft by a factor of about 1.1 to about 4.

In a specific embodiment, the bent portion of the device forms an angle between the shaft and the base portion of greater than 90 degrees. In a more specific embodiment, the angle is from about 90 to about 180 degrees. In an even more specific embodiment, the angle is about 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 degrees.

In a further embodiment, the first grip associates with the first end of the stem at a certain grip angle. In a specific embodiment, the grip angle is about 40 to about 160 degrees. In an even more specific embodiment, the grip angle is about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 degrees.

In another embodiment, the device or a portion thereof, is elastically deformable. The device or a portion thereof may be made of a material or combination of materials that provides a flexural modulus sufficient to allow the first end of the stem to elastically deform in a direction towards the second end of the stem by a deformation distance of from about 0.1 to about 4 inches when a weight is applied to the device. In a specific embodiment, the deformation distance comprises about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 inches. Further the weight applied may include about 1 to about 10 pounds.

In a further embodiment, disclosed is a method for conducting a surgery that implements a disclosed hand stabilizing device embodiment. The method involves inserting a finger into the first grip of the hand stabilizing device and resting the device on a surface proximate to a surgical site.

### Definitions

The present disclosure may be understood more readily by reference to the following detailed description of preferred embodiments of the disclosure as well as to the examples included therein. All numeric values are herein assumed to be modified by the term “about,” whether or not explicitly indicated. The term “about” generally refers to a range of numbers that one of skill in the art would consider equivalent to the recited value (i.e., having the same function or result). In a specific embodiment, the term “about” includes a value that is +/−10% of the stated numerical value. For example, about 40 degrees includes angles of 36 degrees and 44 degrees, and all values in between. In many instances, the term “about” may include numbers that are rounded to the nearest significant figure.

As used herein “microsurgery” refers to any type of surgery requiring a surgical microscope. As used herein “surgical microscope” refers to any magnification device, such as an optical microscope, designed to be used in a surgical setting, typically to perform microsurgery. A surgical microscope typically has a magnification in the range of from about 4× to about 40λ. Microsurgical techniques may be utilized in several specialties, including but not limited to: general surgery, ophthalmology, orthopedic surgery, gynecological surgery, otolaryngology, neurosurgery, maxillofacial surgery, plastic surgery, podiatric surgery, pediatric surgery, oral surgery, dentistry, and endodontics. Oral and maxillofacial surgery (OMS or OMFS) specializes in treating many diseases, injuries and defects in the head, neck, face, jaws and the hard and soft tissues of the oral (mouth) and maxillofacial (jaws and face) region.

As used herein “supermicrosurgery” refers to a microsurgery which allows the anastomosis of smaller caliber vessels and microvascular dissection of vessels having a diameter of less than about 0.8 mm, or of less than about 0.5 mm, such as in an exemplary range of from about 0.3 to about 0.8 mm.

The term “annular aperture” as used herein refers to aperture that comprises at least a portion that is curved. In a specific embodiment, an annular aperture is an enclosed circle.

As used herein “surgical anastomosis” refers to a surgical technique used to make a new connection between body structures. The body structures may carry fluid, such as blood vessels or bowels. The advent of microsurgical techniques have allowed “nerve anastomoses” and operations to restore fertility after tubal ligation or vasectomy. All such techniques are included generally in the term “surgical anastomosis”, but anastomoses of more minute structures performed under a surgical microscope are often referred to as “microanastomosis.” For example, an arterial anastomosis is used in vascular bypass and a colonic anastomosis is used to restore colonic continuity after the resection of colon cancer. A surgical anastomosis can be created using suture sewn by hand, mechanical staplers and biological glues, depending on the circumstances. While an anastomosis may be end-to-end, equally it could be performed side-to-side or end-to-side depending on the circumstances of the required reconstruction or bypass. As used herein “reanastomosis” refers to a surgical reconnection usually reversing a prior surgery to disconnect an anatomical anastomosis, e.g. tubal reversal after tubal ligation.

The term “surgical site” refers to a site in which a surgical procedure is conducted.

The term “proximate to a surgical site” refers to a location at or within a distance to a surgical site about equal to the length of a hand stabilizing device embodiment.

### DESCRIPTION OF ILLUSTRATED EMBODIMENTS

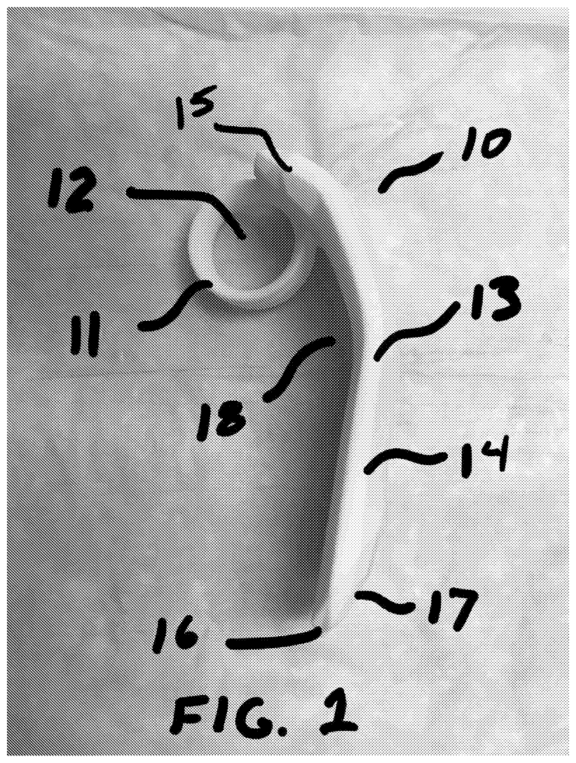

Referring to FIG. 1, a device 10 that may be used as a hand-held surgical stabilizer is shown. The device 10 may include a grip 11, defining an annular aperture 12. The grip 11 may be sized to accommodate a surgeon's finger. The grip 11 may or may not form a complete ring; in other words, the grip 11 may include a gap in its circumference or perimeter. Such a gap may allow the grip 11 to flex and thereby to accommodate various finger sizes. The grip 11 may be generally circular in shape or may be any suitable shape to accommodate a surgeon's finger. The grip 11 may have a diameter within a range having a lower limit and/or an upper limit. The range of the diameter can include or exclude the lower limit and/or the upper limit. The lower limit and/or upper limit can be selected from about 0.5, 0.6, 0.7, 0.8, 0.9, 1, 1.1, 1.2, 1.3, 1.4, 1.5, 1.6, 1.7, 1.8, 1.9, and 2 inches. For example, according to certain embodiments, the grip 11 may have a diameter of from about 0.5 to about 2 inches, from about 1 to about 1.5 inches, or any combination of lower limits and upper limits described.

Still referring to FIG. 1, the device 10 may further include a stem 13. The stem 13 may include a shaft 14, having a first end 15 and a second end 16. The distance from the first end 15 to the second end 16 along the shaft 14 may be within a range having a lower limit and/or an upper limit. The range can include or exclude the lower limit and/or the upper limit. The lower limit and/or upper limit can be selected from about 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, and 10 inches. For example, according to certain embodiments, the distance from the first end 15 to the second end 16 along the shaft 14 may be from about 3 to about 10 inches, from about 5 to about 6 inches, or any combination of lower limits and upper limits described.

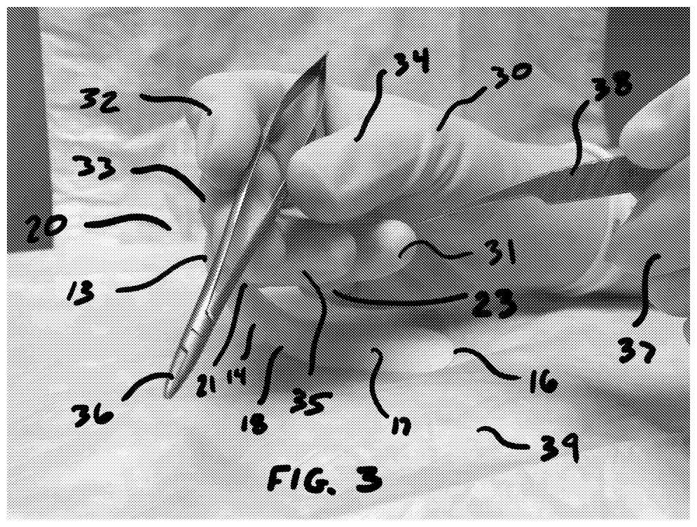

The grip 11 may disposed adjacent to the first end 15 of the stem 13. For example, the grip 11 may be affixed to the grip 11 adjacent to the first end 15. The grip 11 may be affixed integrally or removably to the stem 13. The shaft 14 of stem 13 may include at least one bent portion 18. The at least one bent portion 18 may serve to dispose the second end 16 of the stem 13 toward or beneath the grip 11. The at least one bent portion 18 may also serve as an inflection point for elastic deformation of the shaft 14 according to certain embodiments wherein the shaft 14 is flexible (See FIG. 3). The shaft 14 of the stem 13 may also include a base portion 17 disposed adjacent to the second end 16 of the stem 13. The base portion 17 may extend from second end 16 toward or to the bent portion 18. The base portion may be flattened, tapered, flared, and/or tongue-shaped. The base portion 17 may provide additional surface area for contacting a surface on which the device 10 is rested during use as a surgical stabilizer (See FIG. 3). The additional surface area may provide increased stability.

According to various embodiments hand stabilizing devices 10, having a single grip 11 may be used by a surgeon in a second position. In this second position, the surgeon may move the device 10 completely out of the way by the rotating device 10 around a finger, via the grip 11. The surgeon's hand may then be able to use any tool or perform any action without any obstruction. The hand stabilizing device 10 may, however, remains readily and easily accessible; it being simply rotated behind the surgeon's hand. Thereby various embodiments of the device 10 offer free movement, flexibility and extended reach from any angle the surgeon chooses.

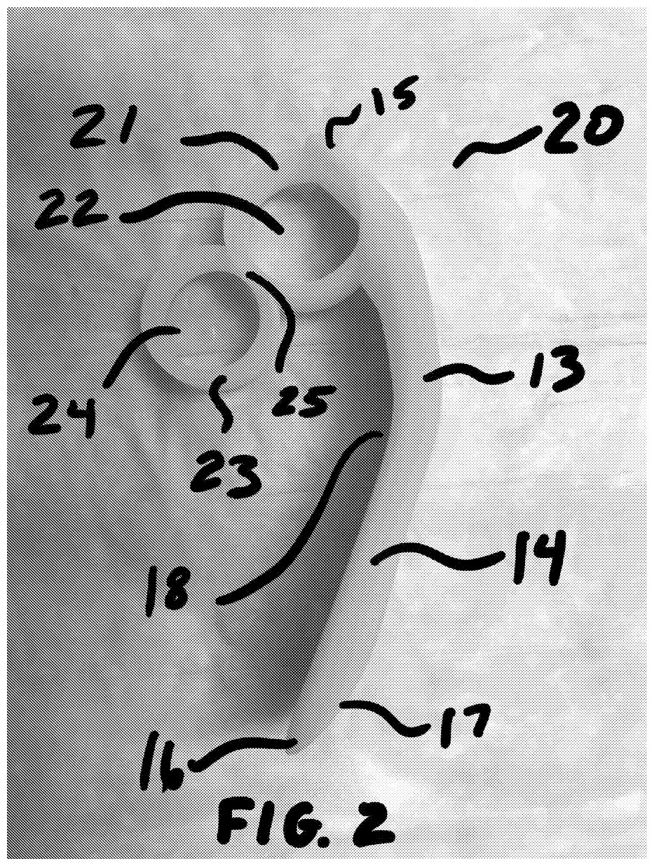

Various other embodiments of device 10 may be readily envisioned, including embodiments including a plurality of grips (See FIG. 2) and/or a plurality of stems, as well as embodiments having multiple bends along the shaft 14.

Still referring to FIG. 1, the device 10 may comprise any suitable material or combination of materials, including metals, such as stainless steel, or plastics, such as thermoplastic polymers, thermosetting polymers. The stem 13 and/or the grip 11 may, for example comprise a metal structure surrounded by a plastic outer layer.

According to various embodiments the device 10 may be elastically deformable. For example, the material(s) of construction may be selected to provide a flexural modulus or a bending modulus sufficient to allow the first end 15 of the stem 13 to elastically deform in a direction towards the second end 16 of the stem 13 by a deformation distance of from about 0.1 to about 4 inches under a load approximately equal to the weight of a surgeon's hand, resting on the device 10, for example via a finger inserted through the grip 11. The precise flexural modulus may be adapted for different applications and/or to accommodate the preferences of a particular surgeon.

The deformation distance may be within a range having a lower limit and/or an upper limit. The range can include or exclude the lower limit and/or the upper limit. The lower limit and/or upper limit can be selected from about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, and 4 inches. For example, according to certain embodiments, the deformation distance may be from about 0.1 to 4 inches, from about 1 to about 3 inches, or any combination of lower limits and upper limits described.

The weight sufficient to cause deformation of the device may be within a range having a lower limit and/or an upper limit. The range can include or exclude the lower limit and/or the upper limit. The lower limit and/or upper limit can be selected from about 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, and 10 pounds. For example, according to certain embodiments, the weight may be from about 1 to about 10 pounds, from about 5.25 to about 8 pounds, or any combination of lower limits and upper limits described.

FIG. 2 shows an embodiment of a device 20, similar to the device 10 illustrated in FIG. 1, but with a first grip 21, defining a first annular aperture 22 and a second grip 23, defining a second annular aperture 24. The first grip 21 may be affixed integrally or removably to the stem 13 at a first end 15. The second grip 23 may be affixed integrally or removably to the first grip 21 at a connection point 25. The description above with respect to device 10 is generally applicable to device 20, similar features being identified with the same reference numerals. Like device 10, device 20 may be used as a hand-held surgical stabilizer. Since device 20 has a plurality of grips, a surgeon may insert multiple fingers into the apertures, which may provide additional stability and comfort for performing certain surgical procedures. The first grip 21 and/or the second grip 23 may or may not form complete rings; in other words, the first grip 21 and/or the second grip 23 may include a gap in their circumference or perimeter. Such a gap may allow either grip to flex and thereby to accommodate various finger sizes. The first grip 21 and/or the second grip 23 may be generally circular in shape or may be any suitable shape to accommodate a surgeon's finger(s).

Still referring to FIG. 2, the first grip 21 may have a diameter within a range having a lower limit and/or an upper limit. The range can include or exclude the lower limit and/or the upper limit. The lower limit and/or upper limit can be selected from about 0.5, 0.6, 0.7, 0.8, 0.9, 1, 1.1, 1.2, 1.3, 1.4, 1.5, 1.6, 1.7, 1.8, 1.9, and 2 inches. For example, according to certain embodiments, the first grip 21 may have a diameter of from about 0.5 to about 2 inches, from about 1 to about 1.5 inches, or any combination of lower limits and upper limits described. The second grip 23 may have a diameter that is the same as, larger than, or smaller than grip 21. For example, the second grip 23 may have a diameter within a range having a lower limit and/or an upper limit. The range can include or exclude the lower limit and/or the upper limit. The lower limit and/or upper limit can be selected from about 0.5, 0.6, 0.7, 0.8, 0.9, 1, 1.1, 1.2, 1.3, 1.4, 1.5, 1.6, 1.7, 1.8, 1.9, and 2 inches. For example, according to certain embodiments, the second grip 23 may have a diameter of from about 0.5 to about 2 inches, from about 1 to about 1.5 inches, or any combination of lower limits and upper limits described.

FIG. 3 shows the device 20, as also shown in FIG. 2, being used by a surgeon. The surgeon's hand 30 interacts with the device 20 by inserting a fifth finger 31 through the grip 23 of the device 20 and by inserting a fourth finger 35 through the grip 21 of the device 20. The surgeon's first finger (or thumb) 34, second finger 32, and third finger 33 are completely free to grip and manipulate a first tool 36. Due to the unobtrusive nature of the device 20, the surgeon's fifth finger 31 and fourth finger 35 are also available to manipulate, to support, or to balance any tool as needed. In this example, the first tool 36 is a surgical instrument, but the tool is not limited to surgical devices and may, for example, be a pen, pencil, or paintbrush. The surgeon's hand 30 is stabilized while using the tool 36 because base portion 17 of device 20 rests securely on a surface 39. As illustrated in FIG. 3, the shaft 14 of stem 13 of device 20 is being elastically deformed at bent portion 18, which serves as an inflection point. The elastic deformation provides flexible, shock-absorbing comfort for the surgeon's hand 30. Due to the unobtrusive size and shape of device 20, the surgeon has ample room to manipulate not only first tool 36, but also a second tool 38 with a second hand 37.

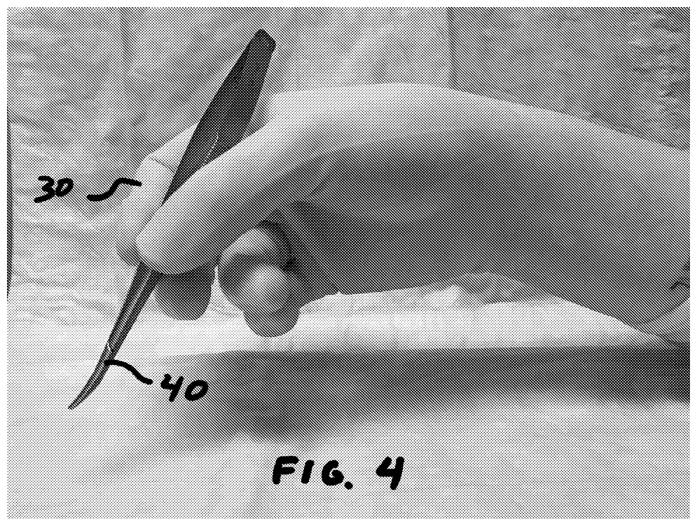

FIG. 4 shows a surgeon's hand 30 using a tool 40 without using a device according to the present invention. As can be seen by comparing FIG. 3 and FIG. 4, devices according to various embodiments of the invention allows surgeons to position their hand as they would without the device without undue encumbrance.

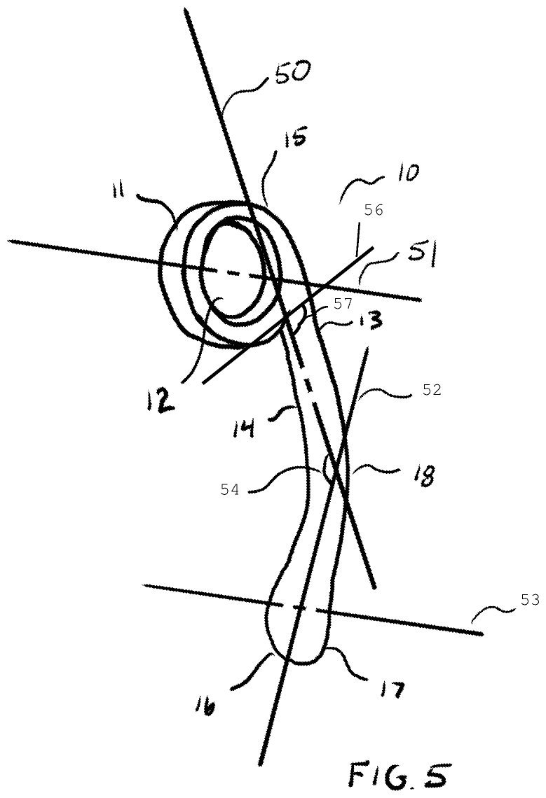

FIG. 5 shows the device 10 in a perspective view. The diagram has been annotated with four axes to illustrate various optional relative positions and relative dimensions of elements of the device 10. A first axis 50 may extend along shaft 14 of stem 13. A second axis 51 may be orthogonally-oriented to grip 11 to extend approximately through the center of aperture 12. A third axis 52 may extend longitudinally along base portion 17 of stem 13 and may intersect the first axis 50 approximately at bent portion 18. The first axis 50 and the third axis 52 may intersect at an angle 54. A fourth axis 53 may extend laterally across the base portion 17, may orthogonally intersect the third axis 52, and may be approximately parallel to the second axis 51.

Still referring to FIG. 5, base portion 17 may extend laterally along the fourth axis 53, which approximately orthogonally intersects the third axis 52, extending longitudinally along the base portion. The base portion 17 may extend laterally along the fourth axis 53 such that the base portion 17 has a width that is greater than a width of the shaft 14 in a direction along the fourth axis 53. The width of the base portion 17 may be from about 1.1 to about 4 times larger than the width of the shaft 14, the widths extending laterally along the fourth axis 53. For example, the width of the base portion 17 may be within a range having a lower limit and/or an upper limit. The range can include or exclude the lower limit and/or the upper limit. The lower limit and/or upper limit can be selected from about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, and 4. Any combination of a lower limit and an upper limit may be used to form a range. In this way, the base portion 17 may be tongue-shaped and/or laterally wider than the shaft 14.

Still referring to FIG. 5, stem 13 may include a bent portion 18. The bent portion 18 may be disposed approximately between the shaft 14 and the base portion 17 of the stem 13. An angle 54 may be present between a first axis 50 extending approximately longitudinally along the shaft 14 and a third axis 52 extending approximately longitudinally along the base portion 17. The angle 54 may be from about 90 to about 180 degrees. For example, the angle 54 may be within a range having a lower limit and/or an upper limit. The range can include or exclude the lower limit and/or the upper limit. The lower limit and/or upper limit can be selected from about 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, and 180 degrees. Any combination of a lower limit and an upper limit may be used to form a range.

Still referring to FIG. 5, various positions of the grip 11 relative to the stem 13 may be envisioned. The grip 11 may be positioned relative to the stem 13 such that a first axis 50 extending longitudinally along shaft 14 of stem 13 and a second axis 51 orthogonally-oriented to grip 11 and extending approximately through the center of aperture 12 intersect or do not intersect. In embodiments where the first axis 50 and the second axis 51 do not intersect, grip 11 may be positioned relative to the stem 13 such that the second axis 51 is on the same side or on the opposite side of the first axis 50.

Still referring to FIG. 5, a fifth axis 56 is shown that intersects axis 50. The fifth axis 56 represents the alignment of the bottom of grip 11 relative to stem 13. At the intersection of axis 50 and 56 is grip angle 57 that represents the angle of the grip 11 relative to stem 13. This angle is shown at about 90 degrees but may range from 40-160 degrees. For example, the grip angle 57 may be within a range having a lower limit and/or an upper limit. The range can include or exclude the lower limit and/or the upper limit. The lower limit and/or upper limit can be selected from about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, and 160 degrees. Any combination of a lower limit and an upper limit may be used to form a range.

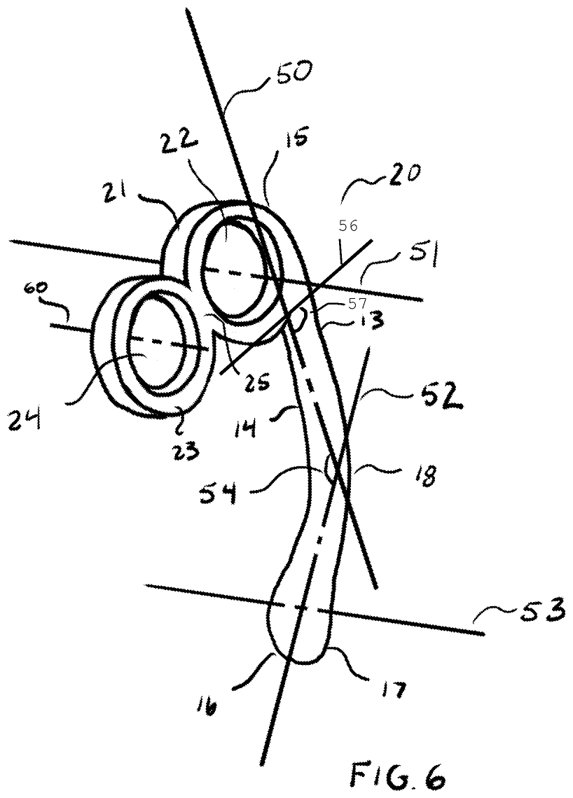

FIG. 6 shows the device 20, as illustrated in FIG. 2, in a perspective view. The diagram has been annotated with six axes, including the five axes previously illustrated in FIG. 5, to illustrate various optional relative positions and relative dimensions of elements of the device 20. FIG. 6 shows an embodiment of a device 20, similar to the device 10 illustrated in FIG. 1 and in FIG. 5, but with a first grip 21, defining a first annular aperture 22 and a second grip 23, defining a second annular aperture 24. The first grip 21 may be affixed integrally or removably to the stem 13 at a first end 15. The second grip 23 may be affixed integrally or removably to the first grip 21 at a connection point 25. The description above with respect to device 10, as illustrated in FIG. 5, is generally applicable to device 20, similar features being identified with the same reference numerals. A sixth axis 60 may be orthogonally-oriented to grip 23 to extend approximately through the center of aperture 24. The sixth axis 60 may be approximately parallel to second axis 51.

Various embodiments provide a new tool to the surgical field that will allow for greater stability and reach without limiting the surgeons' range of motion or ability to place their hand in any location of the surgical area. Various embodiments allow for maximum relaxation of the hand and the ability to grab and handle all surgical tools just the same as without a device. Such embodiments may be useful in all types of surgery, as it can be used in training, stabilization during lengthy surgeries or those with hard to reach area. It will also extend the reach for those with small hands and, even limit tremors. Various embodiments may be particularly beneficial in microsurgery in OMFS cases, as this surgery requires lengthy procedures with extreme precision throughout.

For at least these reasons, the hand stabilizing devices according to various embodiments may aid surgeons in micro-surgery and supermicro-surgery where fine motor skills are paramount. The ergonomics of the device may provide stabilization of the hand as well as improved wrist and forearm range of movement and decreased effort of forearm stabilization muscles. Benefits may include improved accuracy of micro-movements (which may translate to better patient outcomes), improved surgeon comfort and decreased muscular fatigue. For example, various embodiments may improve patient outcome by higher anastomosis success rates, and microanastomosis success rates, may improve patient satisfaction with intricate surgeries (such as cosmetic, paediatric, etc), and may improve surgeon satisfaction through less muscular discomfort and fatigue. The benefits of various embodiments may easily be translated into other situations, such as; young surgeons in training, established surgeons with a tremor and a multitude of intricate surgical procedures in a variety of specialties.

Although the present invention has been described in considerable detail with reference to certain preferred versions thereof, other versions are possible. Therefore, the spirit and scope of the appended claims should not be limited to the description of the preferred versions contained herein.

The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C § 112, sixth paragraph. In particular, the use of “step of” in the claims herein is not intended to invoke the provisions of 35 U.S.C § 112, sixth paragraph.

What is claimed is:

1. A hand stabilizing device comprising:
a stem, having
a shaft,
a bent portion, and
a base portion,
the shaft extending from a first end of the stem to the bent portion,
the base portion extending from a second end of the stem to the bent portion; and
a first grip, associated at the first end of the stem, the first grip defining a first annular aperture.

2. The hand stabilizing device according to claim 1, further comprising a second grip affixed to the first grip, the second grip defining a second annular aperture.

3. The hand stabilizing device according to any of claim 1 or 2, wherein the base portion extends laterally relative to the shaft, such that the base portion is laterally wider than the shaft.

4. The hand stabilizing device according to claim 3, wherein the base portion is laterally wider than the shaft by a factor of about 1.1 to about 4.

5. The hand stabilizing device according to any of claims 1-4, wherein the bent portion forms an angle between the shaft and the base portion of greater than 90 degrees.

6. The hand stabilizing device according to claim 5, wherein the angle is from about 90 to about 180 degrees.

7. The hand stabilizing device according to claim 5, wherein the angle is about 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 degrees.

8. The hand stabilizing device according to any of claims 1-7, wherein an axis of the first grip relative to where the first grip associates at the first end of the stem forms a grip angle.

9. The hand stabilizing device according to claim 8, wherein the grip angle is about 40 to about 160 degrees.

10. The hand stabilizing device of claim 9, wherein the grip angle is about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 degrees.

11. The hand stabilizing device of any of claims 1-10, wherein the device or a portion thereof, is elastically deformable.

12. The hand stabilizing device of claim 11, wherein the device or a portion thereof is made of a material or combination of materials that provides a flexural modulus sufficient to allow the first end of the stem to elastically deform in a direction towards the second end of the stem by a deformation distance of from about 0.1 to about 4 inches when a weight is applied to the device.

13. The hand stabilizing device of claim 12, wherein the deformation distance comprises about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 inches.

14. The hand stabilizing device of claim 12 or 13, wherein the weight applied comprises about 1 to about 10 pounds.

15. A method for conducting a surgery comprising inserting a finger into the first grip of the hand stabilizing device of any of claims 1-14 and resting the device on a surface proximate to a surgical site.
